(19) United States
(12) Patent Application Publication
(10) Pub. No.: US 2008/0069907 A1
(43) Pub. Date: Mar. 20, 2008

(54) Compositions for Cancer Prevention, Treatment, or Amelioration Comprising Papaya Extract

(76) Inventors: Chikao Morimoto, Tokyo (JP); Nam H. Dang, Las Vegas, NV (US)

Correspondence Address: SUGHRUE MION, PLLC, 2100 PENNSYLVANIA AVENUE, N.W., SUITE 800, WASHINGTON, DC, 20037, US

(21) Appl. No.: 11/631,655

(22) PCT Filed: Jul. 6, 2005
(86) PCT No.: PCT/JP05/12880

§ 371 Date: Jun. 13, 2007

Related U.S. Application Data
(60) Provisional application No. 60/585,101, filed on Jul. 6, 2004.

(51) Int. Cl. A61K 36/00

(52) U.S. Cl. 424/725

(57) ABSTRACT

The components of a brew/extract of leaves or other parts of the papaya plant (Carica papaya) or fractionated components thereof are effective in the prevention or treatment of stomach cancer, lung cancer, pancreatic cancer, colon cancer or other solid cancers, or lymphoma, leukemia or other blood cancers, and are very safe with few side effects, making their medical significance extremely high. In addition, food compositions comprising a papaya plant (Carica papaya) extract component as an effective ingredient for the prevention or amelioration of cancer are highly expected to become functional foods contributing to the preservation and promotion of human health.

### TECHNICAL FIELD

The present invention relates to compositions or food compositions for cancer prevention, treatment, or improvement. More specifically, it relates to compositions or food compositions that comprise, as an active ingredient, components extracted by brewing leaves or other parts of the papaya plant (Carica papaya), and that are effective in the prevention, treatment, or improvement of stomach cancer, lung cancer, pancreatic cancer, colon cancer, uterine cancer, ovarian cancer or other solid cancers, or lymphoma, leukemia or other blood cancers.

### BACKGROUND ART

Conventional cancer treatment has been widely done using chemotherapy employing chemotherapeutic agents including cyclophosphamide, busulfan and other alkylating agents; methotrexate, 5-fluorouracil and other agents that inhibit nucleic acid synthesis; and actinomycin D, daunomycin and other antibiotics. However, many of these chemotherapeutic agents are toxic not only to cancer cells but also to normal cells, and their side effects are viewed as problematic. Compared to these chemotherapeutic agents, immunotherapy using immune activators that treat cancer by activating the macrophages and T cells of the immune system has attracted attention as a treatment method having fewer side effects. However, therapeutic agents that are effective in the treatment of cancer and yet have few side effects and a high level of safety have not yet been developed.

### DISCLOSURE OF THE INVENTION

Accordingly, the objective of the present invention is to provide compositions or food compositions for cancer prevention, treatment, or improvement that are highly effective in the treatment and prevention of cancer, and yet have few side effects and a high level of safety.

As a result of exhaustive research regarding the aforementioned objective, the present inventors discovered that components of papaya, preferably one or more extracted papaya components obtained by brewing papaya, have superior effects in the treatment of cancer, and that accordingly, they can become compositions or food compositions for cancer prevention, treatment, or improvement with few side effects and a high level of safety, thus completing the present invention. Accordingly, the present invention relates to compositions or food compositions for cancer prevention, treatment, or improvement that comprise as active ingredients one or more components of the papaya plant (Carica papaya), and preferably one or more components extracted by brewing a part of a papaya plant.

A more detailed explanation of the present invention is provided below.

The present invention uses components of the papaya plant (Carica papaya), preferably components extracted by brewing a part of a papaya plant tissue, as active ingredients. The tissue of the papaya plant may be any of its leaves, roots, stems or fruit, but the leaves are particularly preferable. Preferably, this papaya tissue is dried, and the dried material thus obtained is added to cold water or boiling water, brewed for a long time, and the brew thus obtained is used as an active ingredient. Alternatively, without drying the papaya leaves (or some other tissue), the leaves may be added to cold water or boiling water and brewed. More specifically, papaya leaves, for example, may be left in the sun, normally for one or two days, and dried to obtain the dried material. One to several of these dried leaves are added to cold water or boiling water (normally 400 ml to 3000 ml), and preferably 500 ml to 1000 ml, and brewed for normally two hours to 15 hours, and preferably three hours to 12 hours. The vessel used for brewing is preferably not a metal vessel, but rather a glass, wooden, plastic or other vessel.

A component thus obtained, particularly a component extracted by brewing papaya, comprises the effect of suppressing the proliferation of cancer cells, and can be used as it is as an active ingredient of the composition or food composition according to the present invention. Furthermore, the composition can also be provided as a botanical drug. According to the present invention, this brew may be further subjected to a refining process. The components that comprise the effect of suppressing the proliferation of cancer cells can be fractionated, and the fractionated components thus obtained may be used as an active ingredient. Such fractionated components are, for example, preferably obtained from an extract of papaya leaves that, when subjected to gel filtration chromatography using a gel filtration column filled with cross-linked polyvinyl alcohol gel, the gel filtration column having an exclusion limit molecular weight of 40,000 when pullulan is used as a sample, is eluted in a portion of the eluate equivalent to 50-70 vol. % of the volume of the column. As an example of such gel filtration chromatography, the column used for gel filtration chromatography may be a column that is a stainless-steel tube with an inside diameter of 7.6 mm×length of 500 mm, filled with cross-linked polyvinyl alcohol gel with a grain diameter of 9 μm, and having an exclusion limit molecular weight of 40,000 when pullulan is used as a sample (Shodex Asahipak GS-310 7G) (Showa Denko K. K.). A neutral buffer solution, for example a phosphate buffer solution, may be used as the eluate. A specific example is obtained by dissolving 9.6 g of powdered Dulbecco's PBS (−) “Nissui” (Nihon Pharmaceutical Co., Ltd.) in 1 L of deionized water and autoclaving for 15 minutes at 121° C. to obtain a sterilized buffer solution (pH 7.3 to 7.65).

Under these conditions, when the extract is subjected to gel filtration chromatography, it is possible to obtain those components comprising the effect of suppressing the proliferation of cancer cells by collecting fractionated components obtained by eluting a portion of the eluate equivalent to 50-70 vol. % of the volume of the column. As recited in the subsequent Examples, these fractionated components were put through a gel filtration column similar to that described above, the chromatogram was measured with a differential refractometer (RI) and ultraviolet spectrophotometer (SPD-10AVp), and the approximate calculation of the molecular weight and evaluation of the physical properties of the included components was performed based on a comparison of the molecular weights relative to pullulan and oligosaccharide markers with known molecular weights. Components with molecular weights of 1700, 1000, 700, 600, 400, and 300 were detected by an RI detector. Peaks were detected for molecules (groups) at molecular weights of 1700 and 1000 under UV (260 nm), and were also found at low molecular weights (300 to 700), also under UV (260 nm). Accordingly, the fractionated components comprising anti-tumor effects further refined from the extract of papaya leaves are contained within molecules (groups) with molecular weights of 1700, 1000 and 700 to 300 that absorb UV (260 nm), and the molecule (groups) with molecular weights of 1700, 1000, 700, 600, 400, 300 and 200 that are detectable by RI, each of which are thought to act independently or in combination to achieve the anti-tumor effect.

The brews/extracts thus obtained or further fractionated components may be ingested as they arein order to prevent, treat, or ameliorate cancer. The brews/extracts or fractionated components may also be mixed with appropriate ingredients ordinarily used in drinks and taken as a drink. Alternatively, the brews/extracts or fractionated components may be dried if necessary, and then taken in a powder or tablet or other form after the addition of excipients or other additives ordinarily used in powders, tablets or the like.

In addition, when being stored, the papaya components of brews/extracts, or fractionated components thereof, are preferably stored not in a metal vessel, but rather in a glass, wooden, plastic or other vessel under low-temperature conditions, for example around 4° C.

The present invention's brew/extract components, prepared from, for example, a papaya leaf, or fractionated components thereof, are effective in the prevention or treatment of stomach cancer, lung cancer, pancreatic cancer, liver cancer, colon cancer, uterine cancer, ovarian cancer, neuroblastoma or other solid cancers, or lymphoma, leukemia or other blood cancers. In a preferred embodiment, treatment is administered to those suffering from a disease. In another embodiment, treatment is administered as prophylaxis. The dose of the brew/extract components or fractionated components thereof to be administered will depend on the dosage form, symptoms of the subject, type of cancer or the like. However, for example, when the brew/extract is taken, it is normally preferable to take an amount of 100 ml to 750 ml per day, every day for between one month and three months. When the fractionated components are taken, it is preferable to take an amount of 10 ml to 200 ml per day, every day for between one month and three months.

Also, the present invention provides food compositions that comprise a papaya (Carica papaya) brew/extract as an effective ingredient for preventing or treating cancer. In addition to general foods, a food composition of the present invention may include, for example, a health food, a functional food, a specified health food, a nutrient supplement, an enteral nutrient, and the like, but is not limited to these foods so long as it is effective in preventing or ameliorating cancer. Methods for manufacturing the food compositions are usual techniques known to those skilled in the art. That is, a papaya (Carica papaya) brew/extract component according to the present invention can be combined with an additive acceptable in view of food sanitation, and processed to make a general food, a health food, a functional food, a specified health food, a nutrient supplement, an enteral nutrient, etc. For example, an additive such as a stabilizer, preservative, colorant, perfume, vitamin can be appropriately added to a papaya (Carica papaya) brew/extract component, mixed, and processed by standard methods into a form suitable for a food composition, such as a tablet, pill, granule, powder, capsule, liquid, cream, drink, etc. Furthermore, the food compositions of the present invention include those sold with a description or indication written on the food composition's packaging container and/or in a promotional pamphlet, to the effect that the food composition, and/or an ingredient in the food composition, comprises the effect of preventing, or ameliorating cancer.

### BRIEF DESCRIPTION OF THE DRAWINGS

FIG. 1 depicts graphs showing the anti-tumor effect of a papaya leaf extract according to the present invention on AGS (a stomach cancer cell line: 1000 cells/well and 2000 cells/well, cultured for three days).

FIG. 2 depicts graphs showing the anti-tumor effect of the papaya leaf extract according to the present invention on Capan-1 (a pancreatic cancer cell line: 1000 cells/well and 2000 cells/well, cultured for five days; 40000 cells/well, cultured for four days).

FIG. 3 is a graph showing the anti-tumor effect of the papaya leaf extract according to the present invention on DLD-1 (a colon cancer cell line: 20000 cells/well, cultured for four days).

FIG. 4 is a graph showing the anti-tumor effect of the papaya leaf extract according to the present invention on DOV-13 (ovarian cancer cell line: 3000 cells/well, cultured for two days).

FIG. 5 is a graph showing the anti-tumor effect of a 50-fold-diluted papaya leaf extract according to the present invention on Karpas (a lymphoma cell line).

FIG. 6 depicts graphs showing the anti-tumor effect of the papaya leaf extract according to the present invention on MCF-7 (breast cancer cell line: 2500 cells/well and 7500 cells/well, cultured for six days).

FIG. 7 depicts graphs showing the anti-tumor effect of the papaya leaf extract according to the present invention on T98G (a neuroblastoma cell line: 2000 cells/well and 4000 cells/well, cultured for three days).

FIG. 8 is a graph showing the proliferation suppression effect of the papaya leaf extract according to the present invention on Hela (a uterine cancer cell line).

FIG. 9 depicts graphs showing the proliferation suppression effect of the papaya leaf extract according to the present invention on Karpas (a lymphoma cell line).

FIG. 10 depicts graphs showing the proliferation suppression effect of the papaya leaf extract according to the present invention on CD26 negative Jurkat (T cell leukemia cell line).

FIG. 11 depicts graphs showing the proliferation suppression effect of the papaya leaf extract according to the present invention on CD26 positive Jurkat (T cell leukemia cell line).

FIG. 12 shows the results of measurement of the suppression effect of Jurkat T cell proliferation by components of papaya leaf extract fractionated by gel filtration chromatography.

### BEST MODE FOR CARRYING OUT THE INVENTION

Herein below, the present invention will be specifically described using Examples, however, it is not to be construed as being limited thereto.

### WORKING EXAMPLE 1

### Preparation of an Extract From Papaya Leaves

Papaya leaves were left in the air for approximately one to two days under the sun and thus dried, and approximately 27 g, that is, about one dried papaya leaf, was added to 500 ml of water in a glass vessel, brewed for twelve hours, until the total quantity was 50 ml. The brew thus obtained was filter-sterilized to obtain the papaya extract according to the present invention.

### EXPERIMENTAL EXAMPLE 1

### Test of the Anti-Cancer Effects of Papaya Leaf Extract by the MMT Assay Method

Various types of cancer cells were used to examine the anti-cancer effects of the papaya leaf extract, prepared according to Working Example 1, by means of the MMT assay method (Hansen et al., J. Immunol. Methods 119:203-210, 1989).

(1) Method

Cancer cells from the AGS (stomach cancer cell line), Capan-1 (pancreatic cancer cell line), DLD-1 (colon cancer cell line), Dov-13 (ovarian cancer cell line), Karpas (lymphoma cell line), MCF-7 (breast cancer cell line) and T98G (neuroblastoma cell line) were dispensed into a 96-well microtiter plate and cultured in a RPMI-1640 solution medium containing 10% fetal calf serum, and cultured until confluent. Next, the papaya leaf extract prepared according to Working Example 1 was diluted with culture medium by 20 times (1:20), 150 times (1:150) and 400 times (1:400) respectively, and a volume of 100 μl of the dilute solution was added to the cancer cell lines in each well, which were cultured at 37° C. for two to eight days. Thereafter, 25 μl of 3-(4,5-dimethylthiazole-2-yl)2,5-diphenyl tetrazolium bromide (MTT) was added to each well to make the final concentration 1 mg/ml, and they were cultured at 37° C. for an additional two hours, and then 100 μl of isopropanol containing 0.04 N hydrochloric acid was added. After culturing overnight at 37° C., the absorbance at 570 nm was measured and compared against that of the control to examine just how many of the cells were killed by the papaya leaf extract.

(2) Results

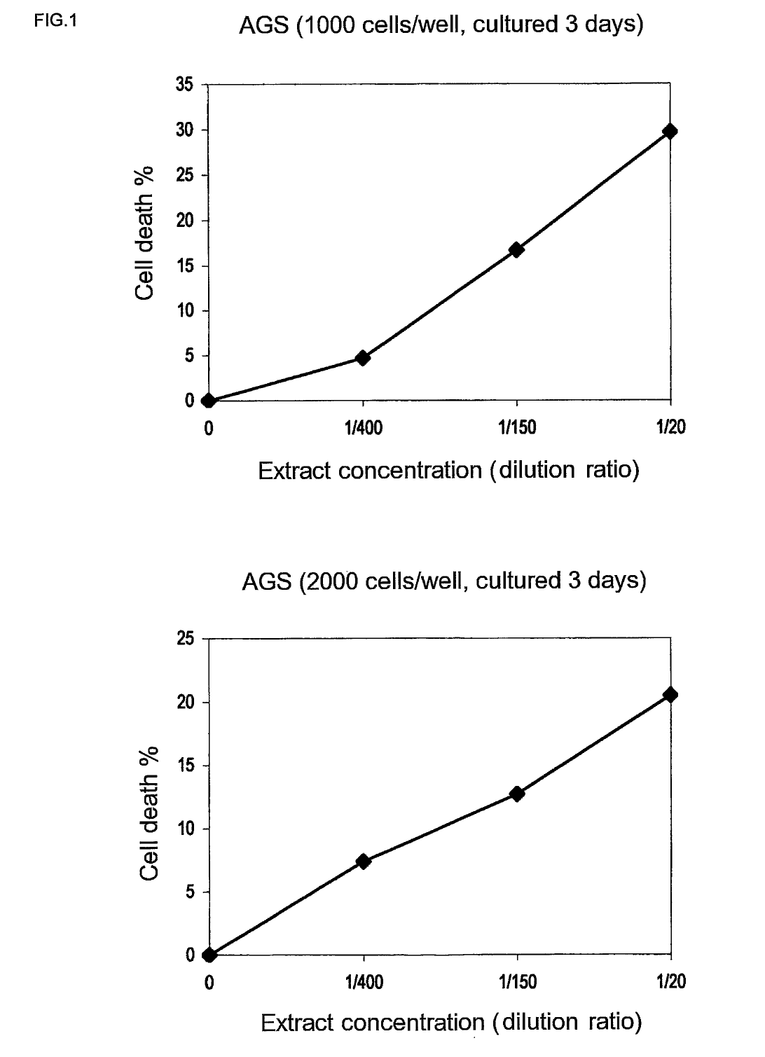

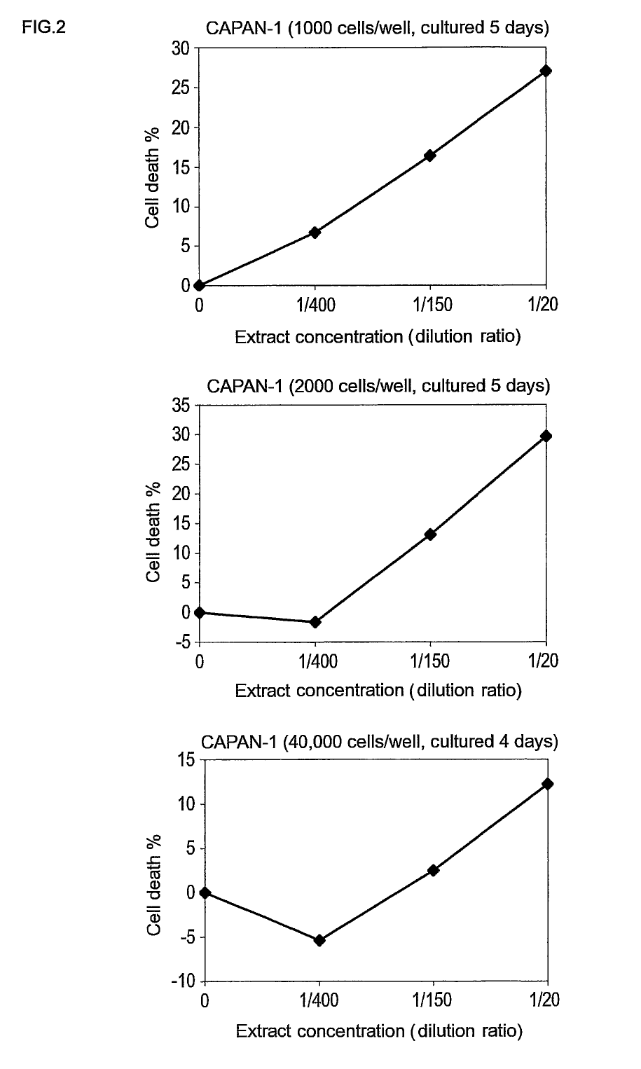

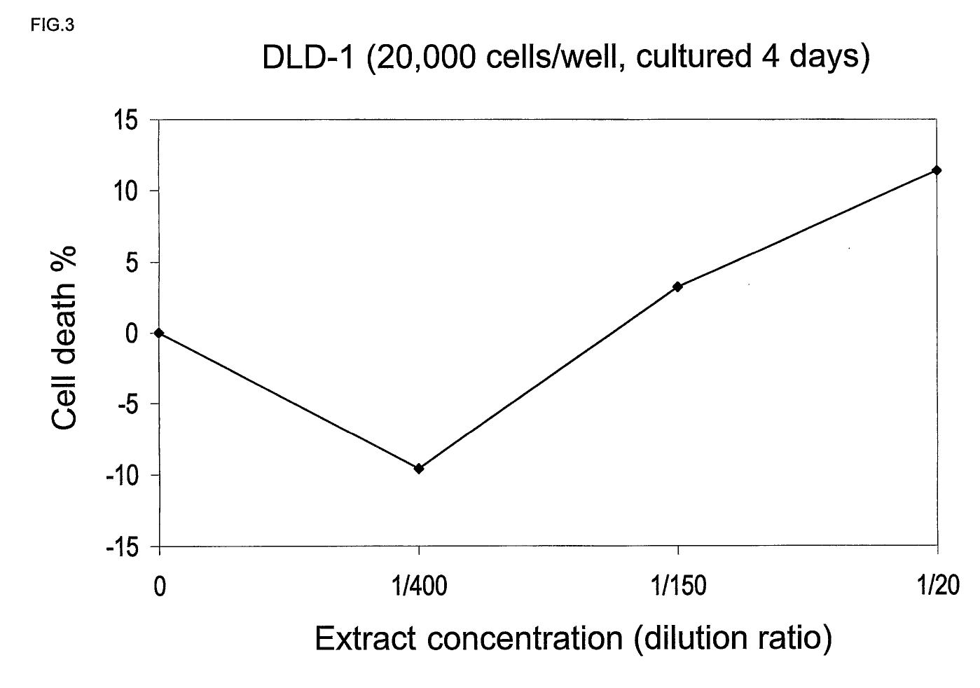

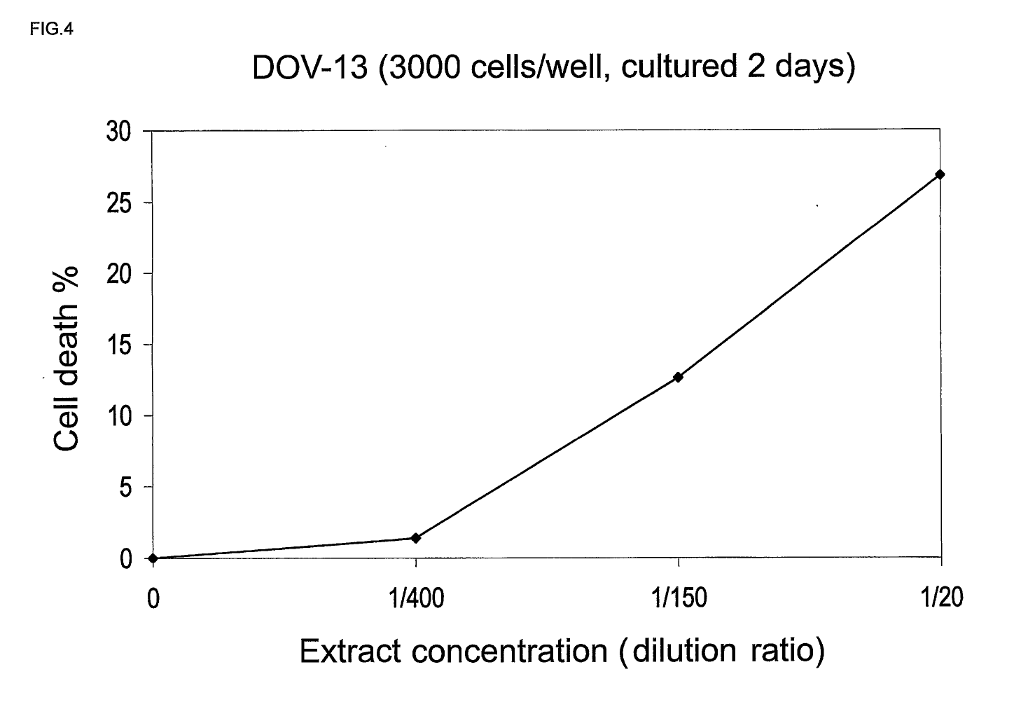

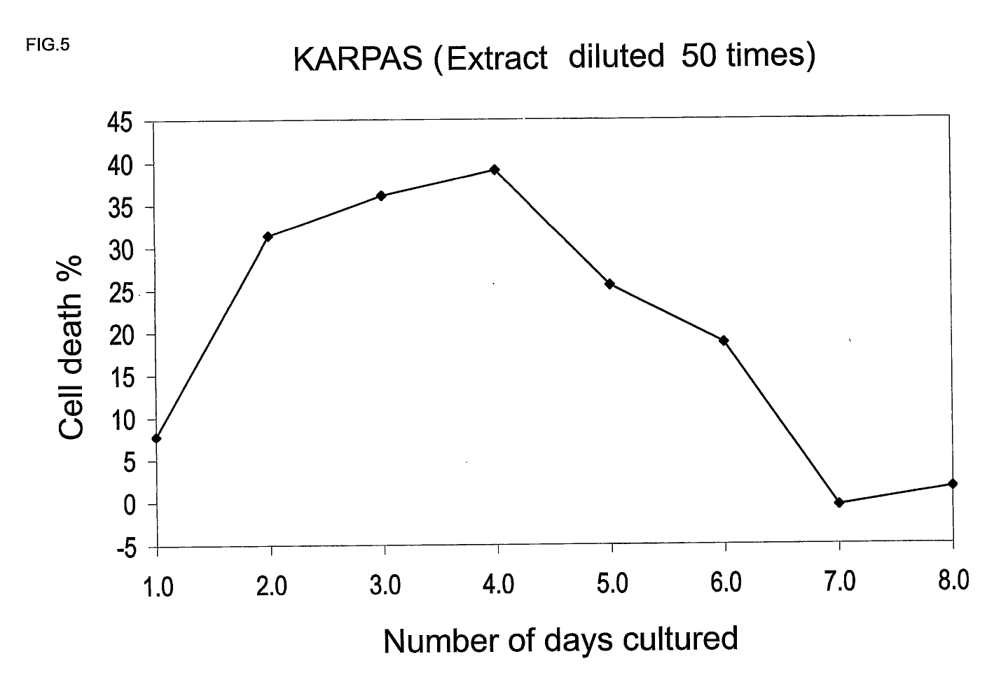

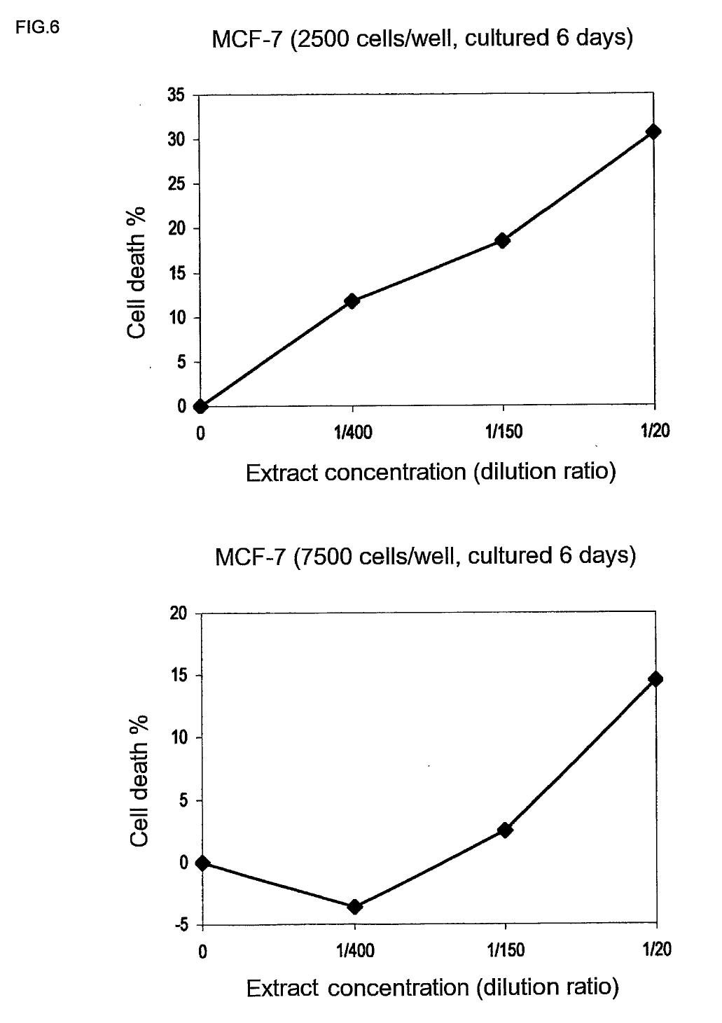

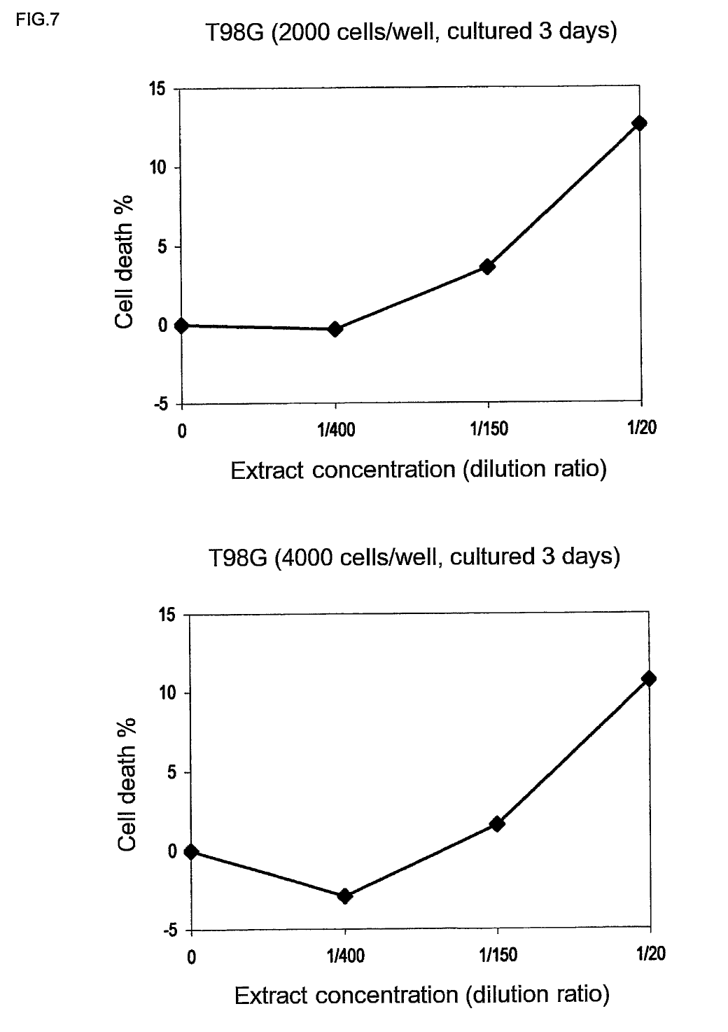

The results thus obtained are shown in FIG. 1 through FIG. 7. As is evident from these figures, the papaya leaf extract had a concentration-dependent anti-cancer effect on each of the cancer cell lines. In particular, as shown in FIG. 5, the maximum value of cell death of 40% appeared on the fourth day of culture in Karpas (lymphoma cell line) at a 50 times dilution of the concentration of the extract.

### EXPERIMENTAL EXAMPLE 2

### Test of the Anti-Cancer Effects of Papaya Leaf Extract by ³H-Thymidine Incorporation

(1) Method

Cancer cells from the Hela (uterine cancer cell line), Karpas (lymphoma cell line), CD26 negative Jurkat (T cell leukemia cell line) and CD26 positive Jurkat (CD26 transfectant T cell leukemia cell line) were dispensed at 5×10³/100 μl into a 96-well microtiter plate. Next, the papaya leaf extract prepared according to Working Example 1 was diluted with culture medium (RPMI-1640 solution containing 10% fetal calf serum) to various dilution ratios, and 10 μl of each was added to the cancer cell lines in each well, which were cultured at 37° C. in a 5% CO₂ incubator. After 24 and 48 hours of culturing, ³H-thymidine was added in the amount of 1 μCi (1 microcurie)/10 μl. After four hours, the amount of ³H-thymidine (cpm) incorporated into the cancer cells for the purpose of DNA synthesis was measured with a scintillation counter to examine the effect of suppressing the proliferation of cancer cells.

(2) Results

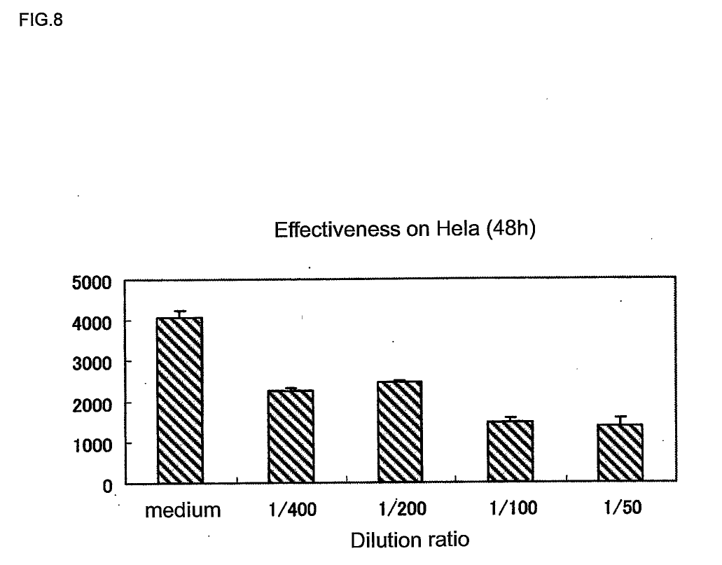

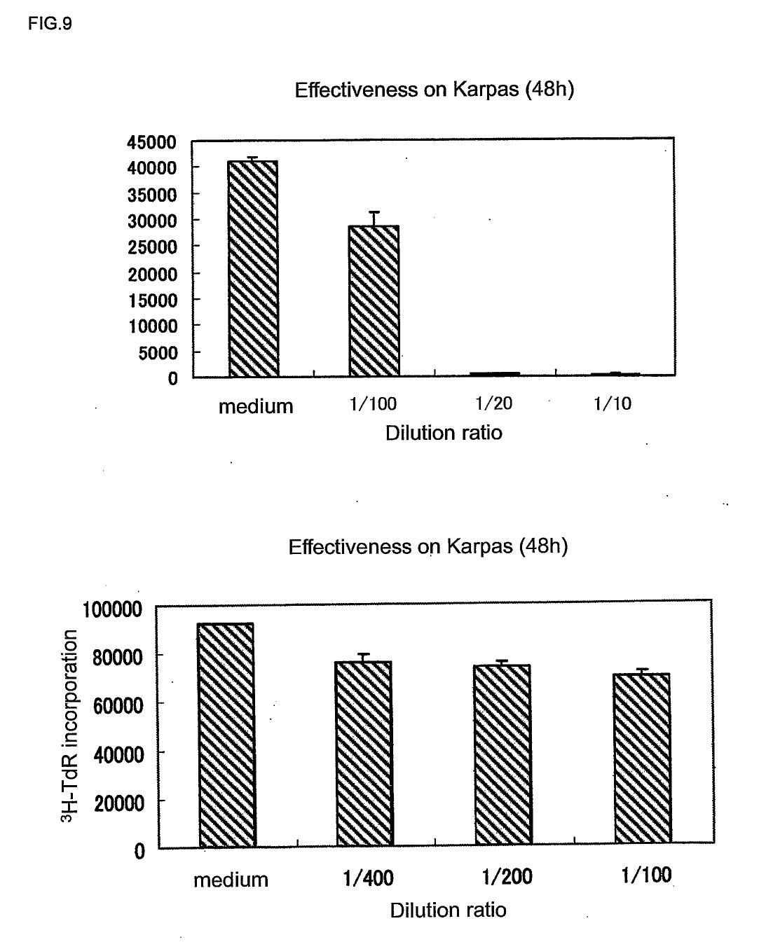

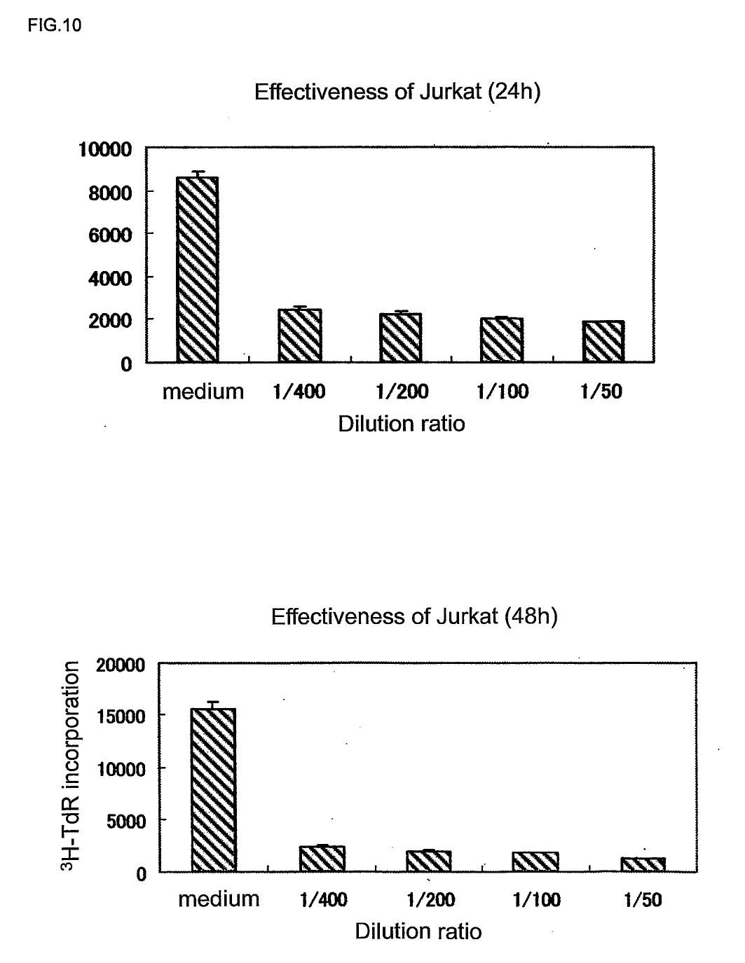

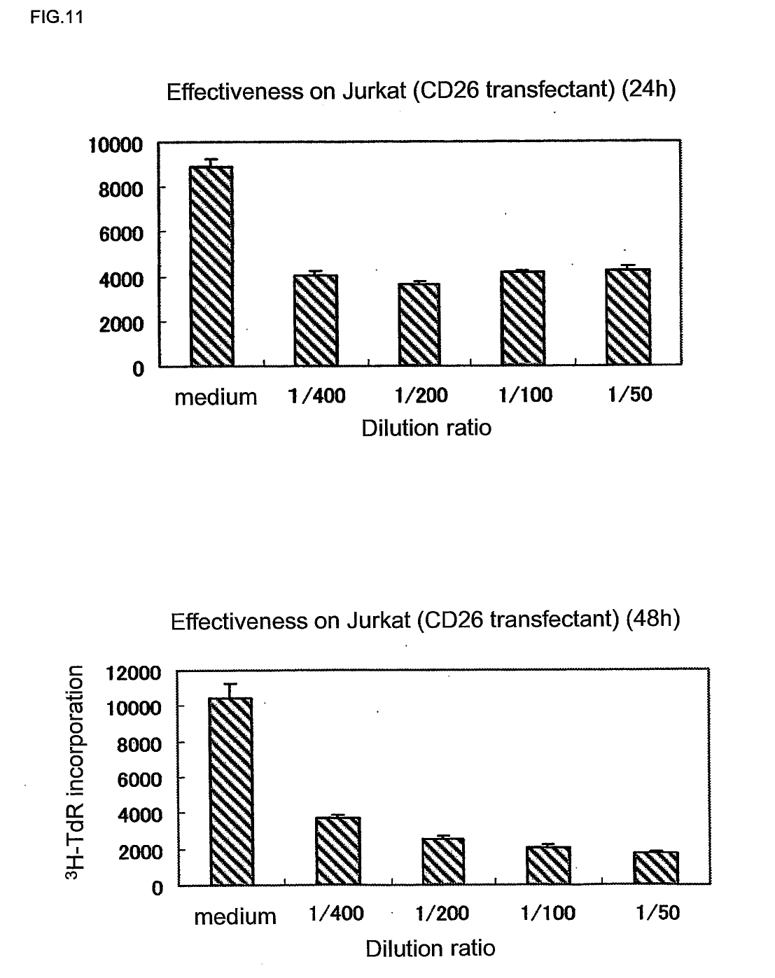

The results thus obtained are shown in FIG. 8 through FIG. 11. As is evident from these figures, the papaya leaf extract suppressed the incorporation of ³H-thymidine by various cancer cell lines, and thus suppressed DNA synthesis. This clearly exhibited a suppressive effect on proliferation.

### EXPERIMENTAL EXAMPLE 3

### Application to Patients

A 47-year-old female patient had stomach cancer that had metastasized to the pancreas. Her tumor marker value CEA was 49 and alpha fetoprotein was 369. One dried papaya leaf was placed in 3000 ml of water and boiled in a wooden vessel for approximately three hours until concentrated to 750 ml. The patient drank approximately 750 ml of the papaya leaf extract every day for 90 days, then took a break from drinking for 90 days, and then continued to drink for 90 days. As a result, the pancreatic metastasis of the stomach cancer disappeared, the CEA level dropped to 2.3, and the alpha fetoprotein also dropped to 2.0, with no relapse found thereafter.

In addition, long-term survival from drinking papaya leaf extract was seen in five lung cancer patients, three stomach cancer patients, three breast cancer patients, one pancreatic cancer patient, one liver cancer patient, and one blood cancer patient.

### WORKING EXAMPLE 2

### Fractionation of Papaya Leaf Extract, Measurement of the Anti-Tumor Effects of Fractionated Components, and Analysis of Fractionated Components

(1) Fractionation by Gel Filtration Chromatography

Dried papaya leaf (approximately 27 g) was heated for one hour to 100° C. in 500 ml of distilled water using a glass beaker, and then further heated for ten hours at 55° C. to prepare an extract concentrated to a volume of 50 ml. Thereafter, fractionation by molecular weight was performed by gel filtration chromatography, and further analysis of the anti-tumor active substances was performed.

Gel filtration chromatography was performed at room temperature using 500 μl of extract. Fluid transport was performed using an LC-10AVp (Shimadzu Corp.), where the moving-phase flow rate was 1 ml/min, and the fractionation method involved manually collecting the eluate from the detector outlet at one-minute intervals, thereby obtaining a total of 60 fractions. Note that the column used for gel filtration chromatography was a column that is a stainless-steel tube with an inside diameter of 7.6 mm×length of 500 mm, filled with cross-linked polyvinyl alcohol gel (grain diameter of 9 μm), and having a molecular weight exclusion limit of 40,000 when pullulan is used as a sample (Shodex Asahipak GS-310 7G) (Showa Denko K. K.). Moreover, the eluate used was obtained by dissolving 9.6 g of powdered Dulbecco's PBS (−) “Nissui” (Nikon Pharmaceutical Co., Ltd.) in 1 L of deionized water, and autoclaving for 15 minutes at 121° C. to obtain a sterilized buffer solution (pH 7.3 to 7.65).

(2) Measurement of the Anti-Tumor Effects of Fractionated Components

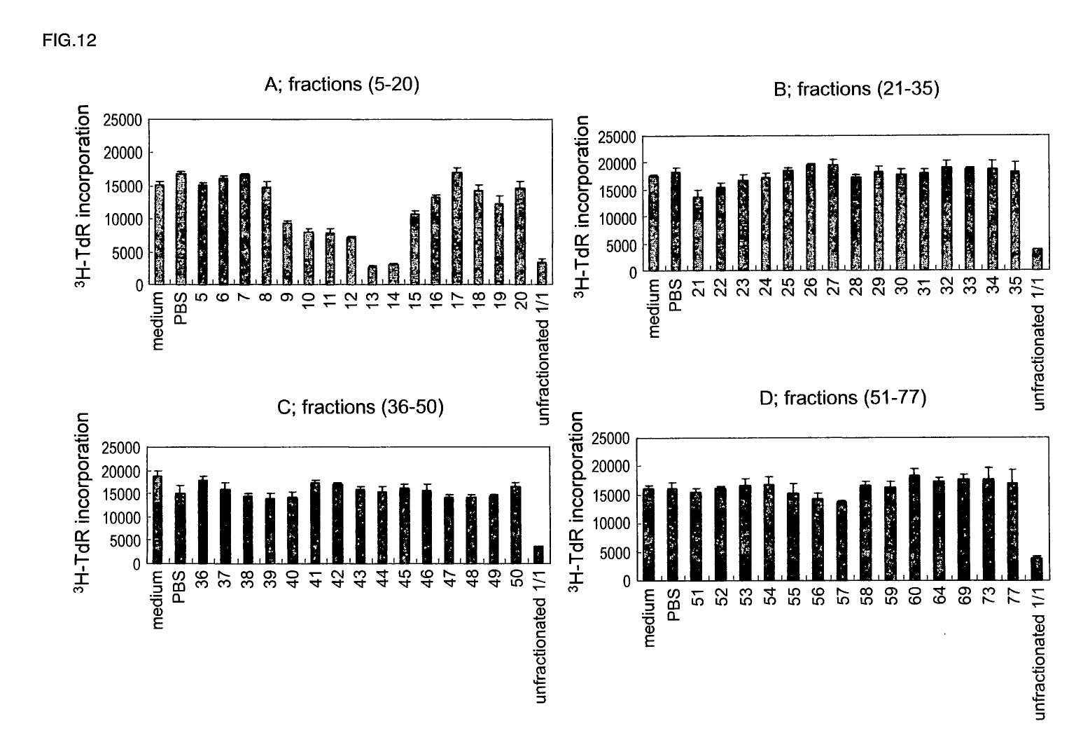

In order to study the anti-tumor effects of the fractionated components, Jurkat T cells were suspended in RPMI-1640 medium (Sigma) (10% fetal bovine serum), and disseminated over a 96-well cell culture microplate (flat bottom) (Becton, Dickinson & Co.) at a concentration of 5×10³ cells/well (5×10⁴ cells/ml). Moreover, the fractionated components obtained by gel filtration chromatography were added to each well in the amount of 10 μl per 100 μl of medium, and culturing was performed. After 48 hours from the commencement of culturing, both the Jurkat T cells in the group with fractionated components added, and the group with no additions, were labeled with ³H-thymidine (³H-TdR) (PerkinElmer Life Sciences) to 1 μCi/well and cultured for four hours. Thereafter, the amount of ³H-TdR incorporated into the cells was measured, and the amount of DNA synthesized by each of the Jurkat T cells was evaluated, thereby examining the anti-tumor effect of each fractionated component. FIG. 12 illustrates the effects of the addition of fractionated components of papaya leaf infusion on the proliferation of Jurkat T cells.

(3) Results

From the results illustrated in FIG. 12, the Jurkat T cells cultured in medium, and Jurkat T cells to which a quantity of PBS equal to the fractionated components was added, exhibited ³H-TdR incorporation of 15192.33±413.4 (cpm) and 16837.33±288 (cpm) respectively. On the other hand, Jurkat T cells added with the fractionated components eluted between 12 minutes and 13 minutes of gel filtration chromatography elution time (fraction 13: the portion of eluate equivalent to 53 to 57 vol. % of the column volume), and the fractionated components eluted between 13 minutes and 14 minutes of gel filtration chromatography elution time (fraction 14: the portion of eluate equivalent to 57 to 62 vol. % of the column volume), exhibited ³H-TdR incorporation of 2757±153.7 (cpm) and 3080±84.7 (cpm) respectively. This is comparable to the ³H-TdR incorporation of 3316.7±516.0 (cpm) by Jurkat T cells to which unfractionated (pre-gel filtration chromatography) extract, 100-fold diluted with culture medium, was added.

Accordingly, the present inventors found that the fractionated components of the papaya leaf extract that comprise the effect of suppressing the proliferation of Jurkat T cells (anti-tumor effect) are present in fractions 13 and 14.

(4) Analysis of Fractionated Components

Furthermore, the fractionated components obtained by gel filtration chromatography that comprise anti-tumor effects (fraction 13 and fraction 14) were each placed in a similar column, and the chromatogram was measured with a differential refractometer (RI) (RID-6A) (Shimadzu Corp.) and ultraviolet spectrophotometer (SPD-10AVp) (Shimadzu Corp.). Approximate calculation of the molecular weight and evaluation of the physical properties of the included components was performed based on a comparison of the molecular weights relative to pullulan and oligosaccharide markers with known molecular weights.

As a result, in fraction 13, components with molecular weights of 1700, 1000, 700 and 300 were detected by an RI detector, and peaks were also detected for molecules (groups) at molecular weights of 1700 and 1000 under UV (260 nm). Thus this fraction is presumed to contain compounds with absorption in these regions. On the other hand, in fraction 14, components with molecular weights of 1700, 1000, 600, 400 and 200 were detected by the RI detector, and peaks were found for molecules (groups) at molecular weights of 1700 and 1000 and also at low molecular weights (300 to 700) under UV (260 nm).

Accordingly, the fractionated components comprising anti-tumor effects extracted from papaya leaves were extracted between 12 minutes and 14 minutes after the start of the gel filtration chromatography used in this experimental system. The components comprising anti-tumor effects are contained within the molecules (groups) with molecular weights of 1700, 1000 and 700 to 300 that absorb UV (260 nm), and the molecules (groups) with molecular weights of 1700, 1000, 700, 600, 400, 300 and 200 that are detectable by RI, each of which are considered to act independently or in combination to achieve anti-tumor effect.

### INDUSTRIAL APPLICABILITY

As described above in detail, the components of an brew/extract of leaves or other parts of the papaya plant (Carica papaya) or fractionated components thereof, for example, are effective in the prevention or treatment of stomach cancer, lung cancer, pancreatic cancer, colon cancer or other solid cancers, or lymphoma, leukemia or other blood cancers, and are very safe with few side effects, making their medical significance extremely high. In addition, food compositions comprising a papaya plant (Carica papaya) extract component as an effective ingredient for the prevention or amelioration of cancer are highly expected to become functional foods contributing to the preservation and promotion of human health.

1. A composition for the prevention, treatment, or amelioration of cancer, comprising as an active ingredient, components extracted by brewing papaya (Carica papaya).

2. The composition according to claim 1, wherein the active ingredient is an extract of papaya leaves.

3. The composition according to claim 1 or 2, wherein the active ingredient is a component derived from an extract of papaya leaves that, when subjected to gel filtration chromatography using a gel filtration column filled with cross-linked polyvinyl alcohol gel, said gel filtration column having an exclusion limit molecular weight of 40,000 when pullulan is used as a sample, is eluted in a portion of the eluate equivalent to 50-70 vol. % of the volume of the column.

4. The composition according to any one of claims 1 to 3 used for the prevention or treatment of solid cancers or blood cancers.

5. The composition according to any one of claims 1 to 4, wherein the composition is in the form of a drink, powder or tablet.

6. A food composition for preventing or ameliorating cancer, comprising as an active ingredient components of a papaya (Carica papaya) extract.

7. The food composition according to claim 6, wherein the active ingredient is an extract of papaya leaves.

8. The food composition according to claim 6 or 7, wherein the active ingredient is a component derived from an extract of papaya leaves that, when subjected to gel filtration chromatography using a gel filtration column filled with cross-linked polyvinyl alcohol gel, the component is eluted in a portion of the eluate equivalent to 50 to 70 vol. % of the volume of the column, wherein the gel filtration column has an exclusion limit molecular weight of 40,000 when pullulan is used as a sample.

9. The food composition according to any one of claims 6 to 8 used for the prevention or amelioration of solid cancers or blood cancers.

10. The food composition according to any one of claims 6 to 9, wherein the food composition is in the form of a drink, powder or tablet.

11. The food composition according to any one of claims 6 to 10, wherein the food composition is a health food, a functional food, a specified health food, a nutrient supplement, or an enteral nutrient.

12. A method for preparing a composition that suppresses the proliferation of cancer cells, the method comprising preparing an extract from leaves or other tissues from a papaya plant, wherein the extract so prepared is a composition that suppresses the proliferation of cancer cells.

13. The method of claim 12, further comprising concentrating the extract.

14. The method of claim 12, further comprising concentrating the extract by at least about two-fold, at least about four-fold or at least about eight-fold.

15. The method of claim 12, wherein preparing the extract comprises brewing the leaves or other tissues from a papaya plant in an aqueous solution.

16. The method of claim 13, wherein the leaves or other tissues from papaya plant are heated in an aqueous solution for about two to about 15 hours.

17. The method of claim 12 further comprising subjecting the extract to column chromatography and collecting an eluted fraction or fractions, wherein at least one eluted fraction so collected is a composition that suppresses the proliferation of cancer cells.

18. A method for preparing a composition that suppresses the proliferation of cancer cells, the method comprising:
(i) preparing an extract from leaves or other tissues from a papaya plant,
(ii) subjecting the extract to column chromatography, and
(iii) collecting an eluted fraction or fractions, wherein at least one eluted fraction so collected is a composition that suppresses the proliferation of cancer cells.

19. The method of claim 17 or 18, wherein the at least one fraction so collected comprises a component detectable by an RI detector and having a molecular weight selected from about 1700, about 1000, about 700, about 600, about 400 and about 300.

20. The method of claim 17 or 18, wherein the at least one fraction so collected comprises a component detectable by a UV detector at 260 nm and having a molecular weight selected from about 1700 and about 1000.

21. The method of claim 17 or 18, wherein the at least one fraction so collected comprises a component detectable by a UV detector at 260 nm and having a molecular weight from about 300 to about 700.

22. The method of claim 17 or 18, wherein subjecting the extract to column chromatography comprises employing a gel filtration column filled with cross-linked polyvinyl alcohol gel.

23. The method of claim 17 or 18, wherein subjecting the extract to column chromatography comprises employing column having an exclusion limit molecular weight of about 2,000 or higher.

24. The method of claim 23, wherein the exclusion limit molecular weight is selected from about 2,000 or higher, about 4,000 or higher, about 10,000 or higher, about 20,000 or higher, and about 40,000 or higher.

25. The method of claim 23, wherein the exclusion limit molecular weight is about 40,000.

26. A composition obtainable by the method of any one of claims 12 to 25.

27. A composition obtained by the method of any one of claims 12 to 25.

28. A method for preventing or treating cancer, the method comprising administering to a subject in need thereof an effective dose of the composition of claim 26 or 27.

29. The method of claim 28, wherein the cancer is selected from the group consisting of stomach cancer, lung cancer, pancreatic cancer, liver cancer, colon cancer, uterine cancer, ovarian cancer, breast cancer, neuroblastoma, lymphoma, and leukemia.

30. A method for preventing or treating cancer, the method comprising administering to a subject in need thereof an effective dose of a composition comprising an extract from leaves or other tissues from a papaya plant, wherein the extract is a composition that suppresses the proliferation of cancer cells.

31. A method for preventing or treating cancer, the method comprising administering to a subject in need thereof an effective dose of a composition comprising a fraction or fractions collected by subjecting an extract from leaves or other tissues from a papaya plant to column chromatography, wherein at least one fraction so collected is a composition that suppresses the proliferation of cancer cells.

32. Use of a component of a papaya extract in the preparation of a medicament for the prevention, treatment, or amelioration of cancer.

33. The use of claim 32, wherein the extract is prepared by brewing papaya.

34. The use of claim 33, wherein the extract is prepared by brewing papaya and then filter-sterilizing it.

35. The use according to claim 32, wherein the extract is an extract of leaves or other tissue of a papaya plant.

36. The use of claim 32, wherein the medicament is used for the prevention, treatment, or amelioration of solid cancers or blood cancers.

37. The use of claim 32, wherein the medicament is in the form of a drink, powder or tablet.

38. The use of claim 32, wherein the medicament further comprises a pharmaceutically acceptable excipient or additive.

39. Use of a component of a papaya extract in the preparation of a food composition for the prevention, treatment, or amelioration of cancer.

40. The use of claim 39, wherein the extract is prepared by brewing papaya.

41. The use of claim 39, wherein the extract is prepared by brewing papaya and then filter-sterilizing it.

42. The use according to claim 39, wherein the extract is an extract of leaves or other tissue of a papaya plant.

43. The use of claim 39, wherein the food composition is used for the prevention, treatment, or amelioration of solid cancers or blood cancers.

44. The use of claim 39, wherein the food composition is in the form of a drink, powder or tablet.

45. The use of claim 39, wherein the food composition further comprises an additive.
